(19) United States
(12) Patent Application Publication
(10) Pub. No.: US 2023/0034413 A1
(43) Pub. Date: Feb. 2, 2023

(54) HIGH FLOW CHECK VALVE FOR MEDICAL GAS APPLICATIONS

(71) Applicant: BeaconMedaes LLC, Rock Hill, SC (US)

(72) Inventors: Mark W. Allen, Rock Hill, SC (US); Paul Barnett, Derbyshire (GB)

(73) Assignee: BeaconMedaes LLC, Rock Hill, SC (US)

(21) Appl. No.: 17/872,460

(22) Filed: Jul. 25, 2022

Related U.S. Application Data
(60) Provisional application No. 63/226,794, filed on Jul. 29, 2021.

(51) Int. Cl. F16K 15/06; A61M 39/24

(52) U.S. Cl. CPC F16K 15/063; A61M 39/24; A61M 2039/2486

(57) ABSTRACT

A check valve for high flow medical gas applications is disclosed. The check valve includes a valve body that defines a flow channel through the valve body from an inlet to an outlet. A movable plunger is in the flow channel of the valve body, with the plunger being constrained in the flow channel between the inlet and the outlet. The plunger comprises a finned frustoconical inlet end a finned frustoconical tip at its outlet which act as flow directing elements which reduce inlet and outlet turbulence at higher medical gas pressures by reducing the gas flow turbulence within the flow channel.

### BACKGROUND

The present invention relates to the distribution of gases using gas networks within a building. In particular, the invention relates to medical gas networks in medical gas facilities such as hospitals and similar facilities.

As those familiar with such facilities are well aware, a number of medical gases (typically including oxygen, nitrous oxide, medical air, instrument air, nitrogen and carbon dioxide) are supplied from a remote location to individual outlets throughout the facility; for example, patient rooms or surgery suites and the like. Most such networks also include a vacuum source for suction and anesthetic gas disposal, along with the necessary piping.

At locations requiring either a beneficial or necessary gas shut off under certain conditions, the relevant hardware is often a check valve; i.e., open under desired gas flow and closed (to prevent loss and leakage) when the gas flow stops.

Medical gas networks must comply with relevant codes. One such code, the NFPA₉₉ health care facilities code requires a minimum flow rate for a given pressure drop expressed as 3.5 standard cubic feet per minute (SCFM) (100 SLPM) with a pressure drop of not more than 5 psi (34 kPa) which ensures the patient has adequate gas flow.

Those familiar with gas flow networks recognize, of course, that a check valve (or for that matter anything that affects flow mechanically) will affect both flow and pressure given that gases (as opposed to liquids) are compressible. These characteristics, which sometimes become problems, are well understood in this art. In particular, in some circumstances the presence of the check valve can make such flow metrics harder to accomplish.

As an example, newer patient rooms or similar spaces provide medical gases as well as power and lighting on modular, ceiling-mounted systems that include rotational joints, connecting arms, and depending columns. These allow a desired gas outlet (or light or power) to be quickly and easily moved into a new position more convenient for the patient’s care or the medical practitioners work. See, e.g., U.S. Pat. No. 7770860.

Using a check valve in such modular systems allows the hoses in the pendant to be removed for service or replaced without the loss of gas or shutdown of the gas system. Current check valve designs, however, tend to create a large pressure drop across the check valve making it hard for the pendant manufacturer to meet the minimum flow rate required by (e.g.) the NFPA99 code.

### SUMMARY

The present invention helps solves the pressure and flow rate problem by making the internal components of the check valve more aerodynamic to improve the flow performance for the same drop in pressure as compared to current check valve designs.

In one aspect, the invention is a check valve for high flow medical gas applications. The check valve includes a valve body that defines a flow channel through the valve body from an inlet to an outlet, and a movable plunger in the flow channel of the valve body. The plunger is constrained in the flow channel between the inlet and the outlet. The plunger comprises a finned frustoconical inlet end having one or more fins (which may be referred to herein as “inlet fins”) and a finned frustoconical tip at its outlet end having one or more fins (which may be referred to herein as “outlet fins”) in order to reduce inlet and outlet turbulence at higher medical gas pressures by reducing the gas flow turbulence within the flow channel.

In another aspect the invention is a method of improving gas flow and avoiding pressure drop in a (medical) gas check valve. The method includes the step of directing an upstream gas flow against a check valve plunger comprising a finned frustoconical inlet end a finned frustoconical tip at its outlet end.

In yet another aspect the invention is a medical gas delivery system. The system includes a facility (hospital) gas supply, a medical gas network between the gas supply and a medical room (patient, operating, etc.) in the facility, a check valve at the medical room and at which the medical gas network terminates, and a medical room outlet downstream of the check valve for medical gas controlled by the check valve. The check valve includes a plunger comprising a finned frustoconical inlet end and a finned frustoconical tip.

The foregoing and other objects and advantages of the invention and the manner in which the same are accomplished will become clearer based on the followed detailed description taken in conjunction with the accompanying drawings.

### BRIEF DESCRIPTION OF THE DRAWINGS

FIG. 1 is a perspective (isometric) view of a check valve according to the invention.

FIG. 2 is a isometric cross-sectional view taken along lines 2-2 of FIG. 1.

FIG. 3 is a side elevational view of the check valve according to the invention.

FIG. 4 is a cross-sectional view of the valve body of the invention taken along lines 4-4 of FIG. 3 and coaxially with the intended flow path.

FIG. 5 is an isometric view of the external face of the orifice cap of a check valve according to the invention.

FIG. 6 is an isometric view of the internal face of the orifice cap of a check valve according to the invention.

FIG. 7 is a cross-sectional view of the orifice cap.

FIGS. 8-10 are isometric views of the plunger in the check valve of the invention.

FIG. 11 is a cross-sectional view of the plunger.

FIG. 12 is an exemplary illustration of a hospital room showing the position of gas outlets in a modular system.

FIG. 13 is a plot of pressure taken against flow rate and showing the performance of the invention against the performance of a conventional check valve.

FIG. 14 is an exploded cross-sectional view of the check valve and spring, and otherwise corresponding to FIG. 14.

### DETAILED DESCRIPTION

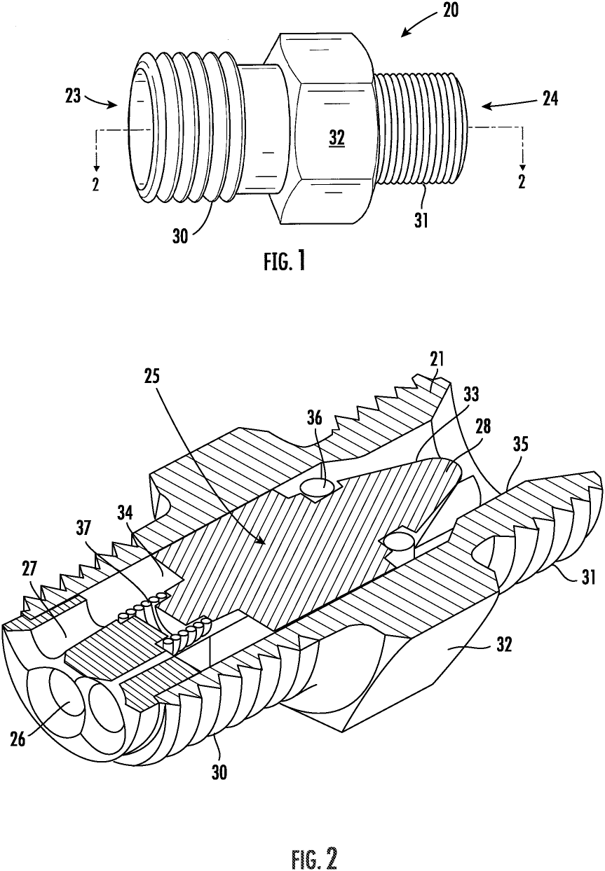

FIG. 1 is a perspective (or isometric) view of the exterior of a check valve 20 for high flow medical gas applications. The check valve 20 includes a valve body 21 that defines a flow channel 22 (e.g., FIG. 4) through the valve body 21 from an inlet 23 to an outlet 24. A movable plunger 25 (FIG. 2) is in the flow channel 22 of the valve body 21. The plunger 25 is constrained in the flow channel 22 by an orifice cap 26 at the inlet 23 of the valve body 21 and by an outlet bevel 35 in the flow channel 22 at the outlet 24 of the valve body 21.

FIG. 1 also illustrates the threaded portions 30 and 31 (male threads are illustrated) typically used to position and connect the check valve in a medical gas network. FIG. 1 illustrates that in exemplary embodiments a nut 32 is either positioned on, or formed integrally with, the valve body 21 to allow an otherwise conventional wrench to turn (typically to tighten or remove) the valve body 21.

FIG. 2 is a perspective cross-sectional view of the valve 20 taken generally along lines 2-2 of FIG. 1. FIG. 2 helps illustrate that the plunger 25 has a finned frustoconical inlet end 34 and a finned frustoconical tip 28 at its outlet end. The fins (i.e. flow-directing elements) on the finned frustoconical inlet end and the fins on the finned frustoconical tip reduce inlet and outlet turbulence at higher medical gas pressures by reducing the gas flow turbulence within the flow channel 22. The plunger 25 includes a beveled shoulder 33 between the finned frustoconical inlet end and the finned frustoconical tip and an O-ring 36 on and coaxial with the long axis (flow direction) of the plunger 25. The O-ring 36 sits against an outlet bevel 35 in the valve body when the check valve 20 is closed. The check valve 20 further incorporates a spring 37 to close the check valve 20 by urging the beveled shoulder 33 of the plunger 25 against the outlet bevel 34 in circumstances under which a gas flow either does not or might not close the check valve 20. The spring 37 is held in place by a retainer 27.

In some embodiments, the check valve 20 may be opened by pushing the plunger 25 towards the spring 37, a task which is typically accomplished by joining the check valve 20 to an intended gas source fixture (not shown).

The orifice cap 26 at the inlet 23 of the valve body 21 helps control gas flow through the check valve 20. As FIG. 2 illustrates, the orifice cap 26 constrains the plunger 25 at the inlet 23 of the valve body 21.

The outlets maybe tubular in geometry and constructed from an elastomeric material that has sufficient plastic memory and strength to either remain closed or reclose itself unless forced open by a sufficient flow of gas. In such check valves the flow of gas in the proper direction will force the lips of the finned frustoconical tip 28 apart so that gas can flow. When the intended gas flow stops, the elastomer collapses to its closed memory position to provide the check function of cutting the gas flow.

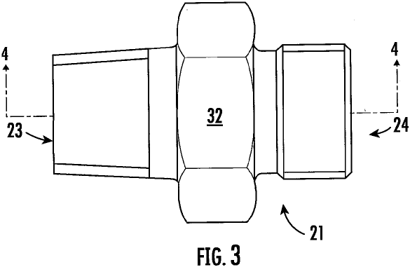

FIG. 3 is a side elevational view of the check valve 20 with commonly numbered items from FIGS. 1 and 2. FIG. 4 is a cross-sectional view taken along line 4-4 of FIG. 3 and in particular shows the outlet bevel 35 as well as the direction of gas flow (arrow “F”) through the valve body 21. In the illustrated embodiment, the outlet bevel 35 forms an angle of about 60° with respect to the direction of gas flow.

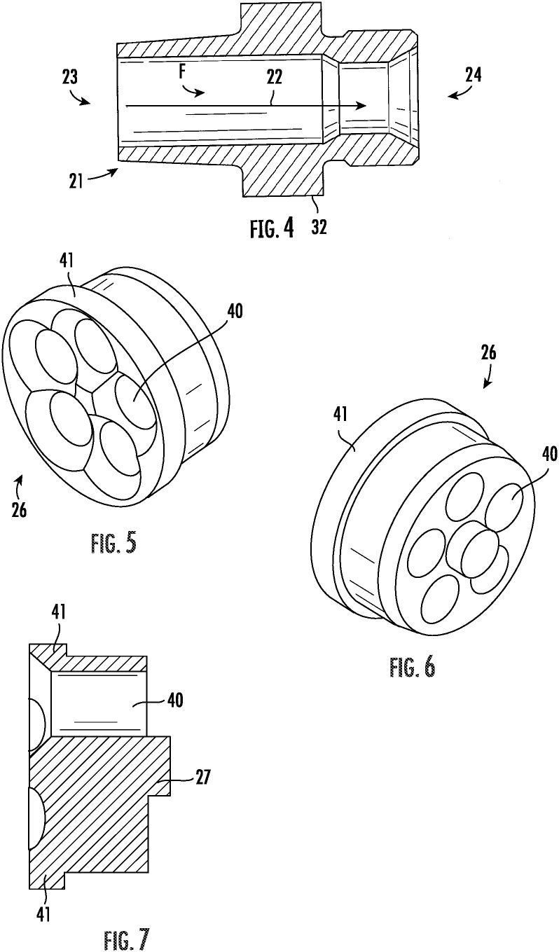

FIGS. 5, 6, and 7 illustrate aspects of an exemplary orifice cap 26. In order to enhance gas flow through the check valve 20, the orifice cap 26 includes a plurality of gas flow passages 40, of which five are present in the illustrated embodiment; however, the plurality of gas flow passages may comprise two gas flow passages, three gas flow passages, four gas flow passages, five gas flow passages, six gas flow passages seven gas flow passages, eight gas flow passages, or more than eight gas flow passages. The passages may comprise anywhere between 10% and 80% of the orifice cap, by volume. FIG. 5 has a perspective orientation from the exterior of the check valve 20, while FIG. 6 shows the orifice cap 26 from the interior perspective. In the illustrated embodiment, a flange 41 orients and positions the orifice cap 26 within the inlet 23 of the valve body 21.

FIG. 7 is a cross-sectional view of the orifice cap 26, the gas flow passages 40, and the flange 41.

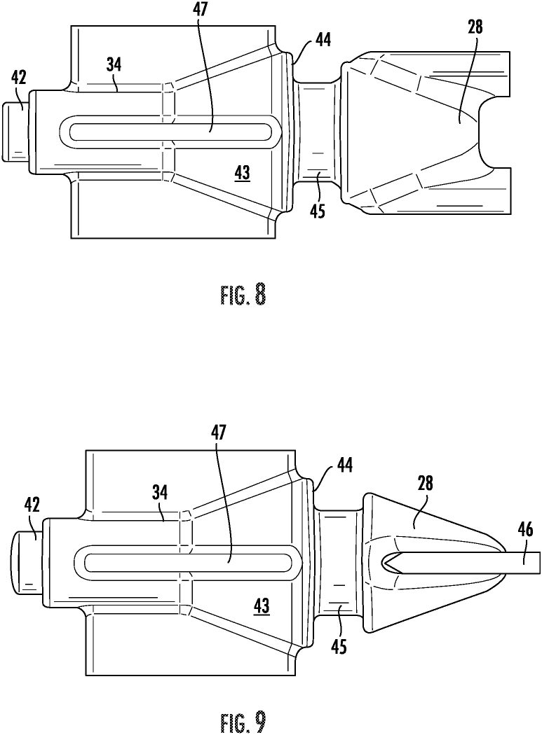

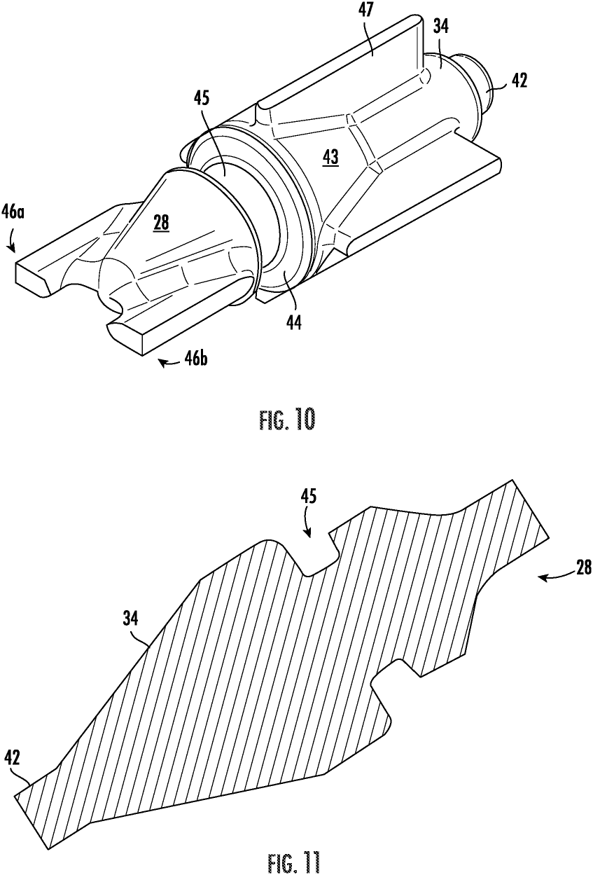

FIGS. 8 through 11 illustrate details of the plunger 25. In addition to the finned frustoconical inlet end 34 having one or more fins 47 and the finned frustoconical tip 28 having one or more fins 46 at its outlet end, the finned frustoconical inlet 34 terminates in a small cylinder 42. In the illustrated embodiment the frustoconical portion 34 terminates towards the mid portion of the plunger 25 in four planar surfaces 43. The planar surfaces 43 terminate in a perpendicular face 44 that together with the remaining portions of the plunger 25 define a channel 45 for the O ring 36. The finned frustoconical tip may comprise any number of outlet fins 46, so long that it comprises at least one fin 46. Further the finned inlet end 34 may comprise any number of inlet fins 47, so long as it comprises at least one fin 47.

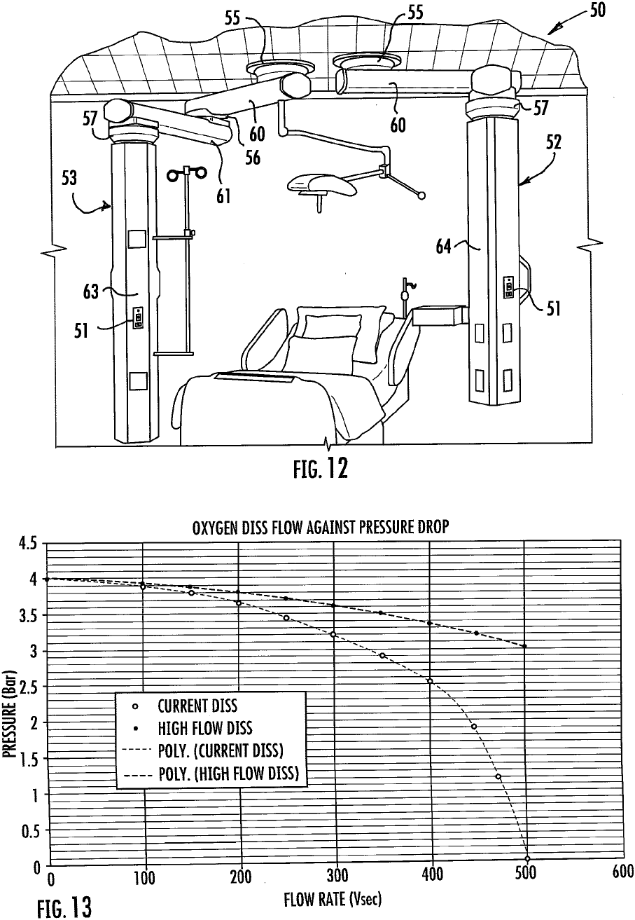

A medical gas delivery system typically includes a facility (e.g., hospital) gas supply, and a medical gas network between the gas supply and a medical room in that facility of which patient rooms, emergency rooms, intensive care units, and operating rooms, are exemplary. FIG. 12 illustrates such a patient room 50 with a plurality of gas outlets 51. In general, the medical gas outlets 51 include corresponding fittings (e.g., DISS, NIST, etc.) and are downstream of the check valve 20 for providing medical gas controlled by the check valve 20.

In FIG. 12, the patient room 50 includes two patient service modules broadly designated at 52 and 53 mounted to the ceiling 54 using rotational joints 55, 56, 57, connecting arms 60 and 61, and pendants 63 and 64. In many cases, the nature of the service modules 52 and 53 are such that the joints provide full 360° rotation which allows the gas outlets, electrical outlets, medical racks, and the like to be positioned quickly and conveniently as desired or necessary.

In a facility such as a hospital, the medical gas network will typically include a plurality of different medical gases, a plurality of the check valves and a plurality of medical gas outlets.

Based on testing to date, the check valve of the invention has a much higher flow rate at a given pressure drop than conventional check valves.

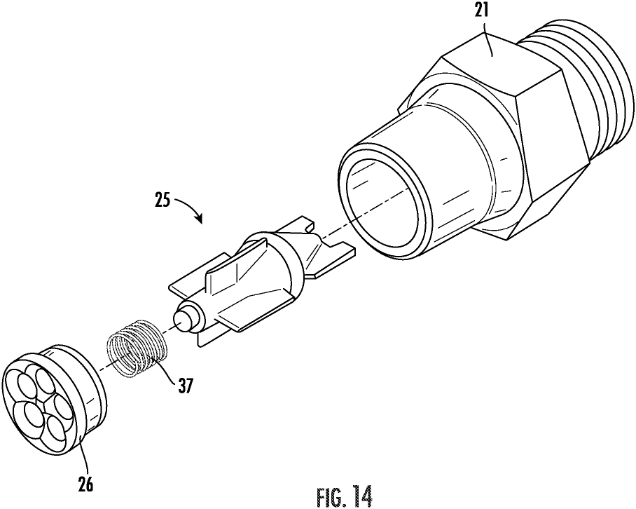

FIG. 13 plots flow rate against pressure drop for a check valve according to the invention and for a conventional check valve. Conventional check valves are well understood and widely available in the medical gas network context, and the inventors submit that the comparison illustrated in FIG. 13 would be similar for a number of conventional check valves.

Thus, in the context of modular or pendant systems (FIG. 12) the check valve 200f the invention allows hoses in the pendants to be removed (e.g., for servicing the hoses or pendants) or replaced without losing gas or requiring a system shut down.

FIG. 13 describes the performance of a “DISS” (Diameter Index Safety System) version of the check valve of the invention. As widely known to skilled persons in both the medical gas network context and the more general health care context, DISS refers to a set of engineering standards that prevent users from linking pressurized gas holding tanks to the wrong outlets, hoses, or tubing. The criteria designate specific-sized connectors and color coded outlet faceplates for each different medical gas. The DISS standards were designed by the Compressed Gas Association (CGA) specifically for medical gases at 200 psig or less. A DISS-compliant system uses unique, gas-specific threaded connections to fit equipment to (e.g.) station outlets.

DISS is not the sole set of standards for connectors, but offers certain functional advantages. Among other authorized hardware for preventing misconnection of gases (or medical air, or vacuum lines), the NIST standards (Non-Interchangeable Screw Threaded) are similarly used to prevent gas connection errors. NIST is, for example, the relevant standard for Britain’s National Health Service. The NIST criteria use a range of male and female components and allocate a set of different diameters and a left- or right-hand screw thread to the joining components for each particular gas.

FIG. 14 illustrates the arrangement of each of the plunger 25, spring 37, orifice cap 26 within the valve body 21.

In another aspect, the invention is a method of improving gas flow and avoiding pressure drop as gases flow through a medical gas check valve. In this aspect, the method comprises directing an upstream gas flow against a check valve plunger that includes a finned frustoconical inlet end and a finned frustoconical tip at its outlet end. The check valve is convenient when disconnecting a downstream fitting from a check valve that incorporates this check valve plunger.

The method also includes improving the gas flow by opening the check valve by connecting the check valve to a corresponding fitting. Exemplary (but not necessarily exclusive) fittings and be selected from the group consisting of DISS-compliant and NIST-compliant fittings.

The method further comprises the step of fixing the check valve in place in a medical gas network prior to the step of directing the upstream gas flow against the plunger.

In the drawings and specification there has been set forth a preferred embodiment of the invention, and although specific terms have been employed, they are used in a generic and descriptive sense only and not for purposes of limitation, the scope of the invention being defined in the claims.

1. A check valve for high flow medical gas applications comprising:
a valve body comprising a valve inlet and a valve outlet, the valve body defining flow channel from said valve inlet to said valve outlet; and
a movable plunger in said flow channel of said valve body;
said plunger being constrained in said flow channel between said inlet and said outlet;
said plunger comprising:
a finned frustoconical inlet end having one or more fins; and
a finned frustoconical tip at its outlet end having one or more fins,
wherein said finned frustoconical inlet end is positioned more closely to said valve inlet than said frustoconical outlet end is positioned to said valve inlet; and
wherein said frustoconical outlet end is positioned more closely to said valve outlet than said finned frustoconical inlet end is positioned to said valve outlet.

2. A check valve according to claim 1, wherein said one or more fins more are axially horizontal.

3. A check valve according to claim 1 wherein said plunger includes a beveled shoulder between said finned frustoconical inlet end and said finned frustoconical tip.

4. A check valve according to claim 2 wherein said plunger further comprises an O-ring on and coaxial with the long axis of said plunger between said finned frustoconical inlet end and said finned frustoconical tip, and in which said O-ring seats against an outlet bevel in said valve body when said check valve is closed.

5. A check valve according to claim 1 wherein said valve body is male threaded at both said inlet and outlet ends so that said check valve can be incorporated into a gas distribution network.

6. A check valve according to claim 5 wherein said male threads are part of a fitting consistent with standards selected from the group consisting of DISS standards and NIST standards.

7. A check valve according to claim 1 further comprising an orifice cap at the inlet end of said valve body for controlling gas flow through said check valve.

8. A check valve according to claim 1 wherein said plunger is constrained in said flow channel by an orifice cap at the inlet end of said valve body and by an outlet bevel in said flow channel at the outlet connection of said valve body.

9. A check valve according to claim 8 wherein said orifice cap has a plurality of gas flow passages.

10. A check valve according to claim 8 further comprising a spring on said plunger for closing said check valve to prevent reverse gas flow.

11. A check valve according to claim 10 in which said spring is positioned on said finned frustoconical inlet end of said plunger for urging said plunger against said outlet bevel.

12. A check valve according to claim 1 that incorporates a nut coaxial with said flow channel for installing said check valve with an appropriate wrench.

13. A method of improving gas flow and avoiding pressure drop in a gas check valve comprising the steps of:
directing an upstream gas flow against a check valve plunger that includes a finned frustoconical inlet end and a finned frustoconical tip at its outlet end.

14. A method according to claim 13 further comprising the step of fixing the check valve in place in a medical gas network prior to the step of directing the upstream gas flow against the plunger.

15. A method of improving gas flow according to claim 13 comprising disconnecting a downstream fitting from a check valve that incorporates the check valve plunger.

16. A method of improving gas flow according to claim 13 further comprising opening the check valve by connecting the check valve to a corresponding fitting selected from the group consisting of DISS compliant and NIST compliant fittings.

17. A medical gas delivery system comprising:
a facility (hospital) gas supply;
a medical gas network between said gas supply and a medical room (patient, operating, etc.) in said facility;
a check valve at said medical room and at which said medical gas network terminates; and
a medical room outlet downstream of said check valve for medical gas controlled by said check valve;
said check valve comprising a plunger, wherein the plunger comprises a finned frustoconical inlet end and a finned frustoconical tip at its outlet end.

18. A medical gas delivery system according to claim 17 further comprising a patient service module downstream of said check valve and upstream of said medical room outlet so that said check valve maintains necessary flow rates at standard pressure drops.

19. A medical gas delivery system according to claim 18 in which said medial gas network includes:
a plurality of different medical gases;
a plurality of said check valves; and
a plurality of said medical room outlets.

20. A medical gas delivery system according to claim 17 wherein said medical room is selected from the group consisting of a patient room, an operating room, an emergency room, and an intensive care unit.
